(19) United States
(12) Patent Application Publication
(10) Pub. No.: US 2011/0207156 A1
(43) Pub. Date: Aug. 25, 2011

(54) Biomarkers for Diagnosis of Breast Cancer

(75) Inventors: Mark W. Duncan, Denver, CO (US); Lindsay Weitzel, Denver, CO (US); Anthony Elias, Denver, CO (US); Timothy Byers, Arvada, CO (US)

(73) Assignee: The Regents of the University of Colorado, Denver, CO (US)

(21) Appl. No.: 13/126,772

(22) PCT Filed: Oct. 29, 2009
(86) PCT No.: PCT/US09/62455

§ 371 Date: Apr. 29, 2011

Related U.S. Application Data
(60) Provisional application No. 61/109,482, filed on Oct. 29, 2008.

(51) Int. Cl. G01N 33/53

(52) U.S. Cl. 435/7.92

(57) ABSTRACT

The present invention provides methods and kits for determining breast cancer. The invention includes the identification and use of biomarkers that are present in different amount or differentially expressed in breast cancer versus normal controls.

### CROSS-REFERENCE TO RELATED APPLICATIONS

This application claims the priority benefit of U.S. Provisional Application No. 61/109,482, filed Oct. 29, 2009, which is incorporated herein by reference in its entirety.

### FIELD OF THE INVENTION

The present invention generally relates to methods and kits for determining breast cancer. The invention includes the identification and use of biomarkers that are differentially expressed or are present in different amounts in breast cancer versus normal controls.

### BACKGROUND OF THE INVENTION

Apart from non-melanoma skin cancer, breast cancer is the most common form of cancer in women. Breast cancer is the number one cause of cancer death in Hispanic women and it is the second most common cause of cancer death in white, black, Asian/Pacific Islander, and American Indian/Alaska Native women. Each year in the US alone about 200,000 women and close to 2,000 men are diagnosed with breast cancer (U.S. Cancer Statistics Working Group. United States Cancer Statistics: 2004 Incidence and Mortality. Atlanta (GA): Department of Health and Human Services, Centers for Disease Control and Prevention, and National Cancer Institute; 2007). According to the American Cancer Society, about 1.3 million women will be diagnosed with breast cancer annually worldwide and about 465,000 will die from the disease. It is estimated that only 5 to 10 percent of breast cancer cases result from inherited mutations or alterations in BRCA1 and BRCA2.

Currently, the best means of reducing the mortality and morbidity associated with breast cancer is early detection and diagnosis because long-term survival rates drop significantly once metastasis has occurred. Identifying breast cancer by mammography is far from optimal: about one third of all women fail to have regular screens and false positive and false negative rates are unacceptably high.

Some biomarkers in tissue are being used as prognostic indicators and as drug targets (e.g., ER and Her2/neu). In addition, biomarkers of tumors are detectable in blood (e.g., CEA, CA15-3, and CA27.29) and can be used to monitor for recurrence. Unfortunately, current biomarker diagnostic methods have limited sensitivity and sensitivity.

Accordingly, there is a need for more sensitive and accurate biomarkers for breast cancer detection.

### SUMMARY OF THE INVENTION

Some aspects of the invention provide a method for determining the presence of breast cancer in a subject. Such methods generally comprise determining the level of a panel of biomarkers in a fluid sample of the subject. Typically, the subject is determined to have breast cancer if the level of biomarkers in the fluid sample of the subject is statistically different from the level of the biomarkers that has been associated with normal controls (e.g., without breast cancer). In some embodiments, the subject is determined to have breast cancer if the level of biomarkers in the patient sample is statistically more similar to the level of the biomarkers that has been associated with breast cancer than the level of the biomarkers that has been associated with the normal controls.

The panel of biomarkers comprises at least three biomarkers selected from the group consisting of SH3 domain binding glutamic acid-rich-like protein; annexin A2; tropomyosin alpha-4 chain; peptidyl-prolyl cis-trans isomerase B; myosin light polypeptide 6; chloride intracellular channel protein 1; Rho GDP-dissociation inhibitor 1; 14-3-3 protein epsilon; coactosin-like protein; lumican; and DJ-1 protein. In some embodiments, the panel of biomarkers comprises DJ-1 protein. In other embodiments, the panel of biomarkers comprises at least four, typically five, and often six biomarkers.

In other embodiments, such methods further comprise comparing the level of biomarkers determined in the fluid sample to a level of biomarkers that has been associated with breast cancer and a level of biomarkers that has been associated with normal controls.

Other aspects of the invention provide a kit for determining the presence of breast cancer. Such kits typically comprise assay kits for determining the level of a panel of biomarkers, where the panel of biomarkers comprises at least three biomarkers selected from the group consisting of: SH3 domain binding glutamic acid-rich-like protein; annexin A2; tropomyosin alpha-4 chain; peptidyl-prolyl cis-trans isomerase B; myosin light polypeptide 6; chloride intracellular channel protein 1; Rho GDP-dissociation inhibitor 1; 14-3-3 protein epsilon; coactosin-like protein; lumican; and DJ-1 protein. Any suitable assay kits known to one skilled in the art can be used, for example, ELISA (single assays or a multiplexed array of analytes), chromatography (e.g., GC/MS, LC/MS, etc.), etc. In some embodiments, such kits comprise assay kits for determining the level of at least three, typically four, often five, and more often six biomarkers. In one particular embodiment, such kits comprise an assay kit for determining the level of DJ-1 protein.

Still other aspects of the invention provide a method for determining the presence of breast cancer in a subject by determining the level of a panel of biomarkers in a fluid sample of the subject. Exemplary fluid samples suitable for methods of the invention include, but are not limited to, whole blood, serum, plasma, urine, saliva, nipple aspirate, tear, etc. In these aspects of the invention, the panel of biomarkers comprises DJ-1 protein and a biomarker selected from the group consisting of SH3 domain binding glutamic acid-rich-like protein; annexin A2; tropomyosin alpha-4 chain; peptidyl-prolyl cis-trans isomerase B; myosin light polypeptide 6; chloride intracellular channel protein 1; Rho GDP-dissociation inhibitor 1; 14-3-3 protein epsilon; coactosin-like protein; lumican; and a combination thereof. In some embodiments, the panel of biomarkers comprises DJ-1 protein and at least two, typically at least three, often at least four, and more often at least five, biomarkers selected from the group consisting of SH3 domain binding glutamic acid-rich-like protein; annexin A2; tropomyosin alpha-4 chain; peptidyl-prolyl cis-trans isomerase B; myosin light polypeptide 6; chloride intracellular channel protein 1; Rho GDP-dissociation inhibitor 1; 14-3-3 protein epsilon; coactosin-like protein; and lumican.

### BRIEF DESCRIPTION OF THE DRAWINGS

FIG. 1 is a representative image of DIGE Gel for one patient. Outlines represent identified proteins that were significantly different in abundance between breast tumor and adjacent normal tissue in 18 patients with ER+/Her-2-negative breast cancer. The numbers correspond to the spot numbers listed in Table 1.

FIG. 2 is a Western blot for peptidyl prolyl cis-trans isomerase B in matched breast cancer and adjacent normal tissues. T: tumor tissue extract; N: matched normal tissue extract; PC: positive control.

FIG. 3 is a Western blot Rho GDP-dissociation inhibitor-1 T: tumor tissue extract; N: matched normal tissue extract; PC: positive control.

FIG. 4 is a graph of pre-op and post-op DJ-1 values for all 17 subjects ordered by number of days between last surgery and post-operative blood draw.

### DETAILED DESCRIPTION OF THE INVENTION

Early detection is known to aid in the discovery of breast tumors when they are of smaller size and have fewer positive lymph nodes. This allows for a higher cure rate. Mammography, while not a perfect screening tool, has been the gold standard for breast cancer screening for decades. Sensitivity of screening mammography is believed to be around 78% and specificity estimates vary from 90-99%. It has been shown that mammographic sensitivity decreases in invasive cancers (73%) and decreases even further in younger women (58%) and in women with dense breasts (44%). Typically, after a suspicious mammogram the next step in the diagnostic process is a biopsy. About 75% of masses biopsied after a mammogram are benign, and mammography misses about 20% of tumors, particularly those that are fast-growing (interval cancers) and those buried in dense breasts. The cumulative risk of a false positive mammogram varies according to the characteristics of each woman, including number of breast biopsies, family history, estrogen use, availability of comparison mammograms, and characteristics of the radiologist. However, by the ninth mammogram cumulative risk can range from 5% in women with low-risk characteristics to nearly 100% in women with high-risk characteristics.

False-negative mammography results lead to a false sense of security that discourages women from seeking medical attention, even after becoming symptomatic. Delays in treatment give the tumor sufficient time to progress and metastasize. There are also consequences of false-positive mammography results. False-positive results take an emotional toll on patients and affect their quality of life. Women who undergo fine needle aspiration, surgical biopsy, or who are placed on early recall after a false positive mammogram, but who are found not to have breast cancer, suffer adverse psychological consequences even months later. In addition, future mammograms may not be as accurate once a woman has undergone breast surgery.

Although several biochemical markers aid in diagnosis of breast cancer, no existing test is sufficiently sensitive and specific for early detection, let alone screening. Without being bound by any theory, it is believed that the molecular diversity of this disease require multi-component panels of markers to provide diagnostic information, but at the same time these panels could also provide predictive and prognostic information for the clinician. A carefully developed array of analytes can aid in early disease diagnosis, sub-classification based on biochemistry, and provide additional guidance to the clinician in selecting the most effective treatment. A comprehensive panel is also useful in monitoring the regression of symptoms, the onset of adverse reactions, and assessing the patient's compliance.

Some of the important factors contributing to prognosis and treatment decisions are tumor stage (comprised of measures of tumor size, nodal involvement, and metastasis), histologic type and histologic grade. However, a small number of biomarkers and clinical assays utilizing both blood and tissue are being used for prognosis, direction of treatment, and surveillance after an initial diagnosis of breast cancer. These include: ER (estrogen receptor, a biomarker found in tissue), Her 2/neu (tissue), CA 15-3 and CA27.29 (both components of MUC 1 found in blood), carcinoembryonic antigen or CEA (blood), and Oncotype Dx (tissue). However, currently these tumor markers are not typically used in screening or diagnosis of breast cancer. Both the progesterone receptor (PR) and Her2 are believed to be prognostic markers of breast cancer. The ER and Her2 are both predictive of response to treatment and are used in directing treatment regimens in breast cancer.

It is believed that since nearly all known biomarkers of cancer are proteins that are either secreted by the tumor into the bloodstream or that exist on the surface of cancerous cells, protein analysis is a logical route by which to discover useful and novel biomarkers. Ultimately, for a change in transcription of a gene to have an effect, that change must result in a corresponding qualitative or quantitative change in the protein the gene encodes (with the exception of genes that produce functional RNA molecules). In addition, because of effects of mRNA alternative processing, truncation and post-transcriptional and translational events (e.g., glycosylation, phosphorylation) etc., there are many more proteins than either genes or transcripts. Some aberrant protein forms may be specific markers of diseases such as breast cancer.

Additional objects, advantages, and novel features of this invention will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### EXAMPLES

### Example 1

Protein levels between tumor and adjacent normal breast tissue from the same breast in 18 women with Stage I/II ER positive/Her-2-neu-negative invasive breast cancer were examined.

Separations were performed in 18 separate Difference Gel Electrophoresis (DIGE) gels (1 gel per patient). After excision and tryptic digestion, matrix-assisted laser desorption/ionization time-of-flight mass spectrometry (MALDI-TOF MS) and peptide mass mapping were used to identify protein spots that were differentially present. The 18 gels were independently replicated. Two candidate biomarkers were verified by western blot analysis.

DIGE showed 243 spots to be differentially abundant between normal and cancer tissues. Fifty protein spots were excised and identified: 41 were over abundant in breast cancer, 9 were less abundant in breast cancers than in normal breast tissue. Western blotting provided independent confirmation for two of the most biologically and statistically interesting candidate biomarkers. Forty-three percent of the proteins found in the duplicate gels were also independently discovered on the original set of 18 gels; 32% of the proteins identified in the original study were verified by the duplicate gel analysis.

Nearly half of the proteins identified as differentially present in breast cancer have not been previously reported as potential biomarkers in the breast cancer literature. Many of the others show promise as potential biomarkers because they have been reported in more than one study and/or associated with pathways known to be related to the disease. Follow-up studies are being conducted to examine some of these proteins as biomarkers.

### Materials and Methods

### Subjects and Specimen Collection

A group of 18 women presenting at the University of Colorado Breast Center newly diagnosed with Stage I/II ER+, Her2/neu− negative, invasive breast cancer were enrolled in this study. Fifty-six percent (10 tumors) of the tumors were classified as T1N0M0, 11% (2 tumors) as T1N1M0, one tumor was TisN0M0. The remaining five tumors were classified one each into the following categories: T1N2M0, T2N0M0, T2N1M0, T2N2M0, and T3N2M0. The average age of the women was 56, the age range was 37 to 80 years.

Patients consented prior to surgery. Tumors had to be larger than 1 cm in size for a patient to be enrolled in this study. The type of breast cancer and receptor status were established from the diagnostic core biopsy. Results of protein over-abundance (i.e., increased levels) by immunohistochemistry (IHC) were used to determine both the ER and Her 2/neu status of the tumors. If IHC results for Her 2/neu were equivocal, FISH analysis was performed.

### Separation of Benign and Cancerous Tissue

Separation of benign and cancerous tissue was performed by a pathologist, by gross examination of the tissue specimen intraoperatively. At least 50 mg of both normal and neoplastic breast tissue was snap frozen and stored at −80 degrees Celsius until analyzed.

### Comparative Proteomics: Difference Gel Electrophoresis (DIGE)

Difference Gel Electrophoresis (DIGE) was performed on the cancerous and benign tissues. Each of the 18 gels contained protein from both benign and cancerous tissues from a single woman.

### Sample Preparation and Protein Extraction

Tissue samples (50 to 350 mg depending on fat content) were homogenized in a buffer comprised of 150 mM NaCl with 50 mM Tris (pH 7.5), 0.3% SDS, and protease inhibitors (Complete™ protease inhibitor cocktail from Roche was used at twice the recommended concentration). The extracts were treated with 200 U/ml of DNAse 1 and 20 U/ml of RNAse A (Sigma-Aldrich, St. Louis, Mo.) and proteins were isolated from tissue samples by methanol/chloroform precipitation. Each dried protein pellet was resolubilized overnight in 400 μL rehydration solution (7M urea, 2M thiourea, 4% w/v CHAPS). Protein determinations were based on the method of Bradford.

### CyDye Labeling—Analytical Gels

Gels used to visualize, match, and analyze (but not pick) protein spots are termed analytical gels. Volumes corresponding to 50 μg of total protein extract from malignant or benign tissue were covalently modified with one of two CyDyes: Cy3 or Cy5 (GE Healthcare, Piscataway, N.J.) as previously reported (Brown et al, 2006). The labeling of cancerous and normal samples was reversed (Cy3/Cy5, Cy5/Cy3) each time a new gel was run to minimize dye bias. After labeling, paired extracts of malignant and benign tissues were combined with 50 μg of Cy2-labeled internal standard (a pool consisting of equal amounts of each benign and malignant extracts) and run on a single 2-D gel.

### CyDye Labeling/Staining—Preparative Gel

Gels used for spot picking are termed preparative gels. The preparative gel was run after differentially abundant spots were identified from analytical gels. The preparative gel contained 50 μg of Cy2-labeled pooled internal standard and 950 μg of unlabeled pooled internal standard. Inclusion of the Cy2-labeled proteins facilitates matching between the preparative gel and the analytical gels because proteins labeled with Cy-dyes tend to migrate differently than unlabeled proteins. The preparative gel was also post-stained with SYPRO Ruby (Molecular Probes) in order to allow visualization of the unlabeled protein spots. Visualizing the labeled and unlabeled protein spots facilitates correct matching.

### First Dimension Separation

First dimension isoelectric focusing was performed on 24-cm immobilized pH gradient strips (IPG 3-10 NL, GE Healthcare). Strips were first incubated in Equilibration Buffer I (10 mins; 50 mM Tris, pH=8.8, 6M urea, 30% glycerol, 20 mg/ml SDS, 50 mM DTT, and bromophenol blue), and then in Equilibration Buffer II (10 mins; 50 mM Tris, pH=8.8, 6M urea, 30% glycerol, and 20 mg/ml SDS, 25 mg/mL iodoacetamide, and bromophenol blue). Strips were then rehydrated for 24 h at 18° C., and focused for 66,000 Vh (analytical gels) or 133,000 Vh (preparative gels).

### Second Dimension Separation

Each strip was placed on an 8-16% gradient polyacrylamide gel (NextGen Sciences, Ann Arbor, Mich.) for second dimension separation at 2 W per gel, 25° C. (Ettan DALT12 Vertical System, GE Health Sciences). Current and voltage were monitored for quality control.

### Gel Imaging

Gels were scanned on a Typhoon 9400 Variable Mode Laser Imager (GE Healthcare) using excitation and emission wavelengths specific to each CyDye (Brown et al., 2006). All gels were analyzed using DeCyder 6.0 Difference In-Gel Electrophoresis (DIGE) Analysis software (GE Healthcare) set to detect 2,000 spots on each gel, excluding the edges of the gel. These spot maps were imported into the Biological Variation Analysis module (GE Healthcare) where all 18 gels were matched on a spot-by-spot basis.

### DIGE Image Interpretation and Statistical Analysis

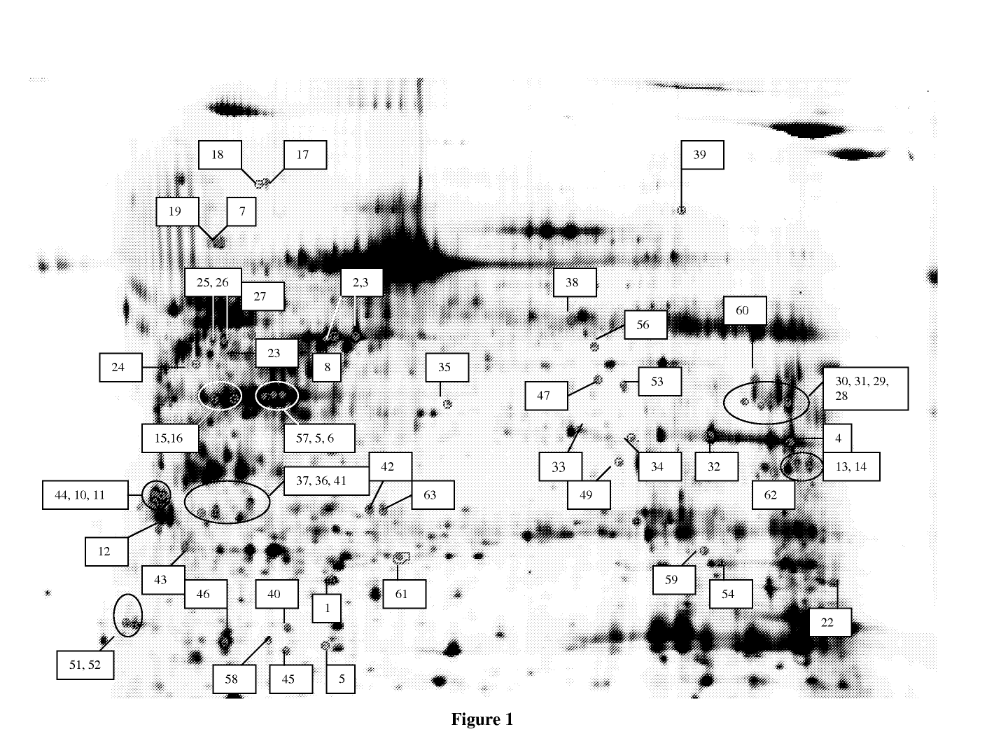

The DIGE image is color-coded to show protein spots that are increased, decreased, or the same in cancerous tissue when compared to benign tissue (FIG. 1). Protein spots were visualized in three dimensions using DeCyder and BVA software (GE Healthcare). This software calculates a fold-change value for each protein as the difference in protein content between the cancer and non-cancer samples.

The spot volume for each protein spot on the gel was calculated from the sum of the pixel intensities within the spot boundary. The background was then subtracted from each spot volume by excluding the lowest 10th percentile pixel value on the spot boundary from all other pixel values within the spot boundary. The spot volume is the sum of these corrected values. Fold-change is the spot volume of the cancerous spot divided by the spot volume of the non-cancerous spot.

A normal distribution was then fitted to the main peak of the histogram displaying all ratios in order to determine a normalization factor. The model curve parameters were then optimized using a least mean square gradient descent algorithm. The normalized distribution was used to identify spot volumes that were significantly different between cancer and non-cancer tissues.

Log standardized protein abundance was then analyzed using the Student's T-test in the DeCyder software testing the null hypothesis that there was no difference in protein abundance between cancerous and non-cancerous samples (or that the average ratio between the two groups is 1). For this study p-values of ≦0.05 were considered statistically significant.

Choosing Proteins to be Identified from the Gel

Proteins that were differentially present in cancer (i.e., under or over abundant) and had p-values≦0.05 were excised from the gel for mass spectral analysis. In addition to these criteria, spots had to have well-defined spot boundaries (i.e., appear to be completely resolved from other nearby components on the gel). Spot excision and in-gel enzymatic digestion were performed by the Ettan Spot Handling Workstation (GE Healthcare). A 2.0 mm picking head was used to excise the spots from the gel. Gel plugs were then transferred to a 96-well plate. In the digester the plugs were washed twice with 100 μL of 50 mM NH₄HCO₃/50% methanol for 5 min, once with 100 μL of 75% acetonitrile for 10 min, and once with 100 μL of 100% acetonitrile for 10 min. Gel plugs were then allowed to dry for 50 minutes, and then trypsin (Promega, San Luis Obispo, Calif.) was added to each well (10 μL, 10 μg/μL in 20 mM NH₄HCO₃), and allowed to digest at room temperature for 16 hours (Rosenfeld et al., 1992). Gel spots were then incubated at 35° C.

### Peptide Mass Mapping and Database Searching

Proteins were identified by peptide mass mapping (PMM) as follows. A solution containing the digested (trypsinized) protein was mixed with matrix solution (5 mg/mL alpha-cyano-4-hydroxycinnamic acid, 0.02% trifluoroacetic acid, 80% acetonitrile), and 0.5 μg of this mixture was spotted onto a matrix-assisted laser desorption/ionization (MALDI) target plate for mass analysis. MALDI-TOF mass spectra were acquired on a voyager DE-STR (Applied Biosystems) mass spectrometer operated in reflectron mode. Peptide mass maps were calibrated to trypsin peaks (m/z 515.33, 842.51, 1,045.56, and 2,211.10). Spectra were processed using ProTS Data (Efeckta Technologies). A peak list was generated and submitted to Mascot (Matrix Science Ltd.) for database searching.

### Western Blotting

The protein extracts (10-20 μg/lane depending on the antibody used) from both normal and cancerous samples were resolved on 4-12% SDS-PAGE gels. The proteins were then transferred to PVDF membranes and blocked for 30 min with 5% non-fat milk in TBS-tween (TBS-T). The blots were incubated in 5% BSA in TBS-T containing either 1:1000 anti-Cyclophilin B (ab3565, Abcam), 1:200 Anti-Rho GDP (ab15198, Abcam), or 1:500 anti-Tropomyosin-4 (ab5449, Abcam) followed by 1:10,000 of the appropriate IgG-HRP-conjugated secondary antibody (anti-rabbit A0168, Sigma for anti-Cyclophilin B and anti-Rho GDP and SC-2305, Santa Cruz Biotechnology for Tropomyosin-4). Protein bands were visualized using TMB substrate (KPL); or for Tropomyosin-4 blots, SuperSignal West Dura Extended Duration Substrate (Thermo Scientific). Rho GDP and Cyclophilin B blots were analyzed using Scion Image software, Tropomyosin-4 blots were analyzed with LabWorks 4.0 Image software. Positive controls used in western blot analysis were human placenta lysate for Rho GDP (ab29745, Abcam), HeLa Nuclear Lysate for Cyclophilin B (ab14655, Abcam), and WI38 human lung fibroblast cell lysate for Tropomyosin-4 (ab3960, Abcam). Paired t-tests were run comparing densitometry values between cancerous and adjacent normal tissue samples.

### Results

### DIGE Results

Approximately 2,000 spots were detected on each of the 18 analytical DIGE gels. Of these, 243 (12%) were differentially present between the two samples and were therefore picked from the gel for identification. Of these, 193 spots that could not be assigned a protein identification were identified as albumin, or were identified as a mixture of more than one protein. Forty-one of the fifty proteins that met the criteria for further study were over abundant in breast cancer tissue, and nine were less abundant in breast cancer tissue (Tables 1 and 2). Average fold-changes for over abundant (selected) proteins range from 1.29 to 3.13; and average fold-changes for less abundant (selected) proteins ranged from −1.42 to −2.29. There were 15 protein spots for which more than one distinct isoform was identified. For all 15 of these proteins, the abundance of all isoforms was in the same direction and of the same magnitude. FIG. 1 shows the placement of the sots on the DIGE gel, spot numbers correspond to those listed in Tables 1 and 2.

### Verification Study

Using the same approach as outlined above, there were approximately 2,000 spots found on each of the verification gels. From the preparative gel 308 spots were picked and 80 identifications were made. Fifteen of the identifications were mixtures of more than one protein, and ten of the spots were albumin. Of the 55 remaining spots, there were a total of 40 distinct proteins. Sixteen (43%) of these identifications were proteins also identified on the original set of 18 gels. Thirty-two percent of the proteins identified in the original study were verified by the duplicate study. Fold-changes and p-values from duplicate gels are listed as ‘verification’ in Tables 1 and 2.

Tables 3 and 4 list biological information for each protein that were relevant when determining the importance of the biomarkers, particularly in blood. This information includes the protein's known biological function, its role or relationship to breast cancer or other cancers, and whether or not it is known to be secreted. FIG. 1 shows the placement of the spots on the DIGE gel, and the spot numbers correspond to those listed in Tables 1 and 2.

### Western Blots

Western blots were performed on paired tumor and normal tissues for three of the proteins identified as potential biomarkers by DIGE. These proteins were peptidyl-prolyl cis-trans isomerase B or Cyclophilin B (PPIaseB), Rho GDP-dissociation inhibitor 1(Rho-GDI alpha), annexin A2 (AA2), and Tropomyosin-4 (TPM4).

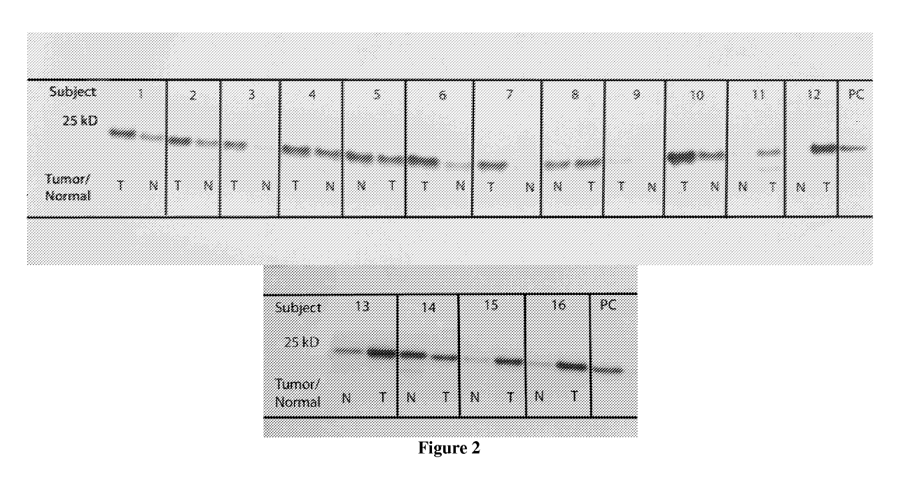

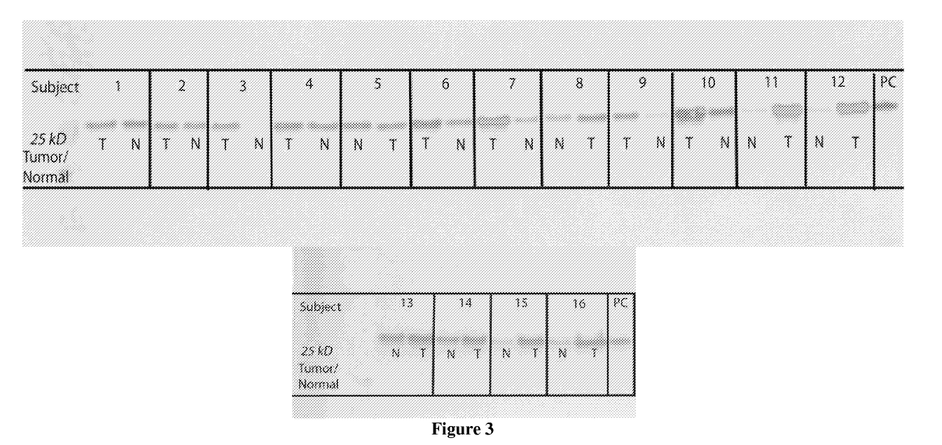

In paired analyses, PPIaseB, Rho-GDI alpha, and TPM4 levels were significantly higher in tumor specimens than in normal specimens (p-values for paired t-test=0.0023, 0.005, and 0.018, respectively). See also Table 5. Western blots for both proteins are shown in FIGS. 2 and 3.

This was a study of proteins that are differentially expressed or are present in different amounts between matched breast tumor and adjacent normal tissues in 18 women with invasive ER+, Her-2/neu− negative breast cancer. At least forty-one proteins were found to be more abundant in breast cancer tissue compared to matched normal tissue, and at least nine proteins were less abundant in breast cancer tissue.

A strength of the DIGE technique is that it allows one skilled in the art to run cancer and normal samples on the same gel. This yields precise relative quantification of protein abundance. However, a limitation to any gel-based protein discovery approach is that there are hundreds of variables and a small number of patients (or gels). This may lead to statistically significant proteins arising by chance alone.

Results from three of the discovered proteins (PPIaseB, RhoGDPI alpha, and TPM4) were verified in the same tissues using antibody-based methodology (western blots). These proteins were chosen for follow-up because they were found on at least two places on the gels (one was also independently verified in the duplicate study); had fold-changes in the range of the top 50% of the significant protein spots discovered; had interesting biology related to breast cancer (yet were fairly novel as biomarkers of breast cancer); had high consistency amongst patients; were known to be secreted; and had antibodies that were commercially available. Both proteins were increased in cancer with high consistency using the western blot method.

Some information that are helpful in determining which proteins are suitable biomarkers of breast cancer include, but not limited to, the magnitude of differential expression or the difference in the amount between tumor and normal tissue, the protein's role in breast cancer biology, whether or not it is known to be secreted, and consistency amongst cancer patients.

### Example 2

Plasma levels of DJ-1 were examined in women who have invasive, early stage, breast cancer.

Using the procedure of Example 1, the present inventors have found that DJ-1 protein was over abundant in breast cancer tissues. Plasma levels of protein DJ-1 were measured by ELISA (Enzyme-Linked ImmunoSorbent Assay) in 48 women with non-metastatic, un-treated invasive breast cancer and 92 controls. These levels were then compared by multiple logistic regression, and sensitivity and specificity were assessed by ROC analysis.

Mean DJ-1 concentrations in plasma were significantly higher in cases than controls (146.5 vs 74.3, p=0.002). The fully adjusted odds ratios for DJ-1 in the second and third tertiles (compared to the first tertile) were 8.7 (CI:1.7 to 42.4) and 57.6 (CI: 11.3 to 291.5). Sensitivity and specificity for elevated DJ-1 as a marker of invasive breast cancer at levels optimized by the ROC analysis were 75% and 84%.

Candidate Protein Discovery from Breast Cancer Tissues

Eighteen women with invasive ER-positive/Her-2-negative breast cancer were used in the example. Cancerous and benign tissues were separated by a pathologist and snap-frozen directly outside the operating room. The details of the laboratory methodology are described above. In brief, after purification, the proteins were tagged with florescent dyes, then paired extracts of malignant and benign tissues were subjected to 2D-DIGE (2-Dimensional Gel Electrophoresis) to achieve a 2-dimensional separation of proteins. Gels were then scanned on a laser imager (GE Healthcare) using excitation and emission wavelengths specific to each dye. Readings from the gels were then analyzed using DeCyder software (GE Healthcare) to detect 2,000 separate spots on each gel. These spot maps were then assessed by the Biological Variation Analysis module (GE Healthcare) to assess differences between proteins from normal breast tissue and breast cancer tissue across the 18 subjects. Normalized spot volumes were compared using a Student's t-tests (2-tailed). Proteins with p-values<0.05 were excised by the Ettan Spot Handling Workstation (GE Healthcare) and identified by mass spectrometry (243 spots). Of these, 41 proteins were identified as more abundant in breast cancer tissue compared to those in normal control. Eleven of these proteins were ranked as most interesting for follow-up in circulation because of their interesting biology related to breast cancer; had high consistency amongst study patients; were known to be secreted; and had antibodies that were commercially available.

Of these, an ELISA kit was commercially readily available for DJ-1 protein. Accordingly, the levels of DJ-1 in the circulation were examined via a case-control study. Cases were 48 women with newly diagnosed Stage I/II invasive breast cancer. Plasma from breast cancer cases was taken before surgery for removal of their newly diagnosed tumor. Seventeen cases also had blood samples drawn approximately six weeks post-operatively (before chemotherapy was initiated) for a pre-operative/post-operative comparison. Control subjects were women without known breast cancer. Plasma from breast cancer patients and controls was collected in glass tubes containing sodium citrate. Blood tubes were spun within two hours of blood collection, and plasma was then stored at −80° C. All specimens were handled using identical procedures for all study subjects.

### Assessment of DJ-1 in Plasma

Enzyme-linked immunosorbent assay (ELISA) was performed using the CircuLex™ Human DJ-1/PARK7 ELISA Kit (Cat. No. CY-9050, MBL International). The standard curve for each ELISA was obtained using the recombinant human DJ-1/PARK7 standard provided in the ELISA kit at concentrations of 100, 50, 25, 12.5, 6.25, 3.13, and 1.56 ng/mL. All kit instructions were followed. Absorbance was read at dual wavelengths of 450/595 using a spectrophotometric microplate reader. Pre-operative and post-operative samples from each patient were placed on the same plate. Antibodies were tested for masking effect by diluting plasma from two separate patients 1:10, 1:20, and 1:40 and running each in triplicate wells. Non-specific binding was also tested with bovine serum albumin run in triplicate at 50 ng/mL.

All samples were assayed in triplicate with both cases and controls included together in each 96-well plate. Pre-operative and post-operative samples from each patient were also placed on the same plate. The limit of detection for this assay was defined as the mean of three blanks plus three standard deviations of the absorbance of the blank as suggested by the kit manufacturer.

### Statistical Analysis

The mean of three triplicate values was used as the value for each subject. A logistic regression was performed using the DJ-1 values as the dependent variable and case/control status as the independent variable. A multiple logistic regression was also carried out by sequentially adding four other cofactors (age, menopausal status, and history of hormone replacement therapy use). An ROC analysis was also performed in order to determine positive and negative values and the optimal cut-off between breast cancer cases and control subjects.

In order to determine a cut-off value using the ROC analysis, various graphs were generated plotting the estimated probability that each DJ-1 value would be categorized into the breast cancer category (described from here on as probability) against: 1) the ratio of the true positive fraction to false positive fraction (described from here on as RATIO), 2) positive predictive value 3) negative predictive value, 4) 1-specificity, 5) sensitivity, and 6) specificity. The estimated probabilities serve as cut-points for predicting the response. The DJ-1 value that corresponded to the probability that optimized the above parameters was chosen as the cut-off between breast cancer cases and controls. All statistical analyses were performed using SAS 9.1 (Cary, N.C.).

### Results

The limit of detection (sensitivity) for the Circulex DJ-1/PARK7 ELISA assay was 0.052 ng/mL and the intra-assay variation was 4.9%. Bovine serum albumin at a concentration of 50 ng/mL was below the limit of detection, and in the masking experiment absorbance values decreased with each decrease in DJ-1 concentration, suggesting that non-specific binding was not significant.

The mean DJ-1 value for cases with breast cancer (n=48) was 146.5 ng/mL and the mean value for controls (n=92) was 74.2 ng/ml (Table 7). For cases, the average length of time between diagnostic biopsy and pre-operative blood draw was seventeen days (median=13 days). There was no statistical relationship between the number of days counted between breast biopsy and blood draw and a breast cancer patient's DJ-1 level (p=0.61).

Tumor grade, ER status, and PR status were all significantly related to level of DJ-1 in plasma. There were two extreme observations in the control group. The first was a control subject whose DJ-1 value was very high (699.5 ng/mL). This subject was 49 years old, and had not had a mammogram in three years, and another 47-year old who had a value below detection. There was also one extreme observation in the case group, a woman with levels several fold higher than the average for cases (784.0 ng/mL). This woman developed widespread metastasis within 6 months of diagnosis. Since removing these extreme observations did not change the results of the study, all observations were included in this analysis.

DJ-1 was analyzed in tertiles (determined by values from subjects in the entire study—cases and controls) and assessed as a predictor of breast cancer status in four different models (Table 8). The odds ratios for breast cancer for the second and third tertile of DJ-1 levels (compared to the lowest tertile) were 8.7 and 57.6 for the fully adjusted model.

An ROC analysis was performed to determine the cut-off value for DJ-1 that most accurately discriminates between breast cancer cases and control subjects. Using the SAS OUTROC output, the DJ-1 cut-off value of 89.0 ng/mL optimized the parameters of sensitivity and specificity. Values for sensitivity (69 to 79%) and specificity (80 to 85%) and area under the curve (81 to 85%) are shown in Table 9. The odds ratio for the fully adjusted model for DJ-1 values above 89.0 is 23.7 (CI: 8.6 to 65.2, p<0.0001).

### Discussion

The present inventors have discovered that there were higher levels of protein DJ-1 in the plasma of women with newly-diagnosed, untreated, breast cancer with no evidence of metastasis. While DJ-1 was first identified as an oncogene involved in cellular transformation via ras-related signal transduction pathways, most studies of DJ-1 as a serum marker have focused on Parkinson's disease (PD).

Protein DJ-1 has been shown to be a negative regulator of PTEN (phosphatase and tensin homolog deleted on chromosome 10), a tumor suppressor involved in the regulation of the phosphatidylinositol 3-kinase (PI3K) signaling pathway. DJ-1 is also known to have a role in antioxidative stress to prevent cell death and has been found to play a role in transcriptional regulation.

In this study, there were two women from the breast cancer group who recurred quickly (within about 6 months) after treatment and who quickly developed widespread metastatic disease. Both of these women had DJ-1 levels higher than average for breast cancer cases at the time of their original diagnosis (784.0 ng/mL and 203.6 ng/mL compared to the study average of 146.5 ng/mL). This indicates that the level of this protein is predictive of severity of disease despite the fact that there was no relationship between plasma DJ-1 and tumor size or nodal status in this study. It should be noted that DJ-1 was higher in the plasma of women with ER-negative tumors.

Plasma DJ-1 (as well as other biomarkers disclosed herein) can be used in the surveillance of patients after removal of (or after treatment for) breast cancer. In addition, plasma DJ-1 can be used in patients who fall into BIRADS (Breast Imaging Reporting and Data System) categories that yield uncertain results and often lead to negative biopsies. These are BIRADS categories 3 (probably benign, 6 month follow-up) and 4 (suspicious abnormality, not characteristic of breast cancer, but biopsy should be considered).

### Example III

Plasma levels of DJ-1 were examined in a group of women before and after removal of an invasive breast tumor.

Plasma levels of protein DJ-1 were measured by ELISA (Enzyme-Linked ImmunoSorbent Assay) in 17 women with non-metastatic, invasive breast cancer before, and again after surgery for removal of their breast tumor. These levels were then compared in paired analysis and predictors of which direction the value changed after surgery were evaluated.

A paired t-test yielded no significant difference between pre-operative and post-operative DJ-1 levels in the samples in this small sample set. However, values for all but one subject changed by more than the referent control subject. Half of the subjects' values decreased putting them within the healthy range after surgery. However, having experienced two surgeries or radiation therapy before the post-operative blood draw was associated with an increase in DJ-1 post-operatively.

### Discussion

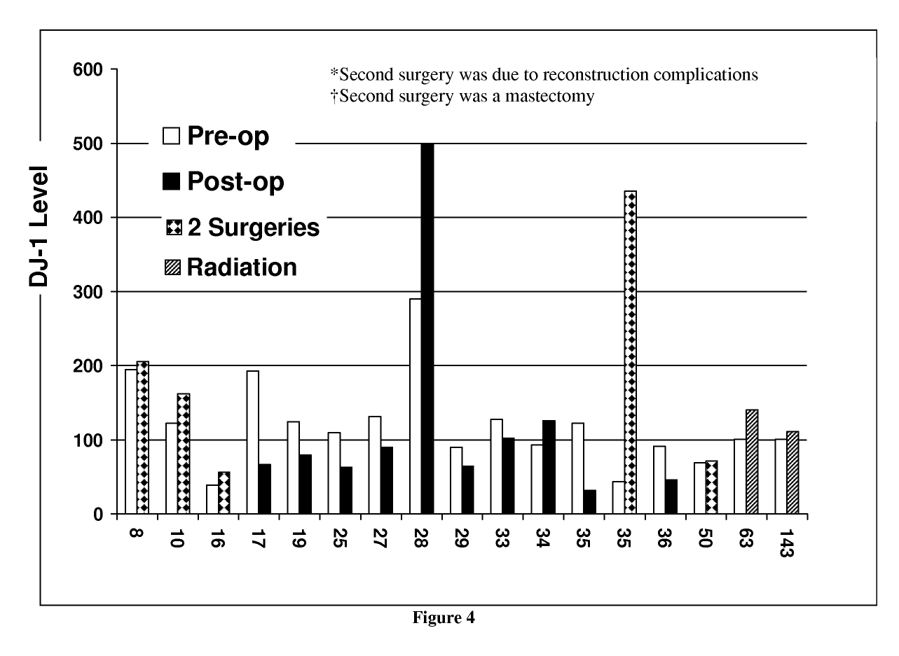

In general, if a subject had a single surgery to remove her tumor, and did not have radiation treatment prior to her second blood sample, her DJ-1 levels decreased from pre-operative levels. For the purposes of discussion, the values pertaining to days out from surgery date in FIG. 4 was used as subject numbers when referring to particular subjects. DJ-1 values for eight subjects decreased after surgery, while values for nine subjects did not decrease significantly. One of the values that did not decrease after surgery (listed in FIG. 4 as 50 days post-op), essentially remained the same.

Subjects Whose DJ-1 Values Decreased after Removal of their Tumor

All eight of the subjects whose values decreased after surgery began with DJ-1 values above the cut-off of 89.0 ng/mL calculated in the sensitivity and specificity analysis in the previous case-control study. Seven of these eight women (88%) reached values in the healthy range after removal of their tumor. None of these subjects received radiation or had a second surgery before their post-operative blood sample was drawn. Samples in this group were all drawn between 17 and 36 days after surgery.

Subject Whose DJ-1 Values Remained the Same after Removal of a Breast Tumor

The subject whose post-operative blood sample was drawn 50 days after removal of her tumor had a post-operative DJ-1 value that increased by a value less than the specified 8.2 ng/mL (69.0 ng/mL to 71.4 ng/mL); therefore for discussion purposes her value will be considered to have remained the same. This was the only subject whose second surgery was for a reconstruction complication, not re-excision purposes. It is also interesting to note that her original DJ-1 value did not put her above the cut-off of 89.0 ng/mL that was determined in the case-control study to differentiate accurately between breast cancer and control subjects.

Subjects Whose DJ-1 Values Did not Decrease after Removal of their Tumor

All eight remaining subjects had post-operative DJ-1 values that were higher than their pre-operative values. Six of these eight subjects (75%) underwent either a second surgery or radiation before their post-operative blood sample was drawn (four underwent a second surgery and two underwent radiation). All second operations in this group were re-excisions for the purpose of removing remaining cancerous tissue. Only one of the surgeries was a mastectomy, this subject's post-operative DJ-1 value increased the most (post-op 35 days, value increased 10-fold) out of all 17 subjects. Samples in this group were drawn between 8 and 143 days after surgery. It is believed that in some instances increased DJ-1 levels post-operatively, or in individuals receiving radiation therapy, may be related to a tissue damage/wound recovery phenomenon.

In addition to time allowed to heal, a couple of other timeframes were calculated to determine if they had an affect on DJ-1 levels. The number of days the tumor was left in the subject's body after the pre-operative blood draw was not related to the direction of plasma DJ-1 change after surgery. Another timeframe whose effect was tested on pre-operative DJ-1 values and found to be insignificant was the timeframe between biopsy and pre-operative blood draw.

The two subjects who received radiation before their post-operative sample was drawn simply had a late blood draw due to scheduling difficulties. They were not known to have residual disease, nor is there any reason to consider their burden of cancer to be higher than the women whose values decreased. It is possible that there may be something about radiation therapy, whether due to an epithelialization process or not, that causes an increase in DJ-1 levels. When tumor markers are used in surveillance after treatment they are most appropriately used after a patient has finished all chemotherapy and radiation.

The situation is different for the subjects who had two surgeries. These subjects underwent re-excision (one underwent a full mastectomy) due to the fact that they had residual disease. Therefore, looking at the timeframe the subjects were enrolled in this study, cancer was present in the re-excision group for a longer period of time than for the women who had a single surgery that removed the tumor in its entirety. Also, FIG. 4 shows that there was a tendency for these women to have had their blood sample taken closer to their last surgery than the women who only had a single surgery; therefore, there was less time for clearance of a tumor marker from circulation.

### Example IV

Biomarkers of breast cancer in plasma were determined.

In the first phase (discovery phase) of this study, at least 41 proteins were found to be significantly over-abundant and nine proteins were found to be under-abundant in the breast cancer tissues of eighteen women when compared to their adjacent normal breast tissue.

Some of the criteria used in selecting proteins for validation via western blot and/or followed-up in plasma were: the magnitude of the difference between cancer and normal tissues, the protein's role in cancer (especially breast cancer) biology, whether or not the protein was known to be secreted, its consistency among subjects, and spot volume in benign tissue. Much of these criteria were organized in table format for proteins of the most biological interest (see Tables 13a and 13b).

Table 13a is written from the viewpoint that a biomarker with lower abundance in benign tissue is more specific. Yet some may have more confidence in a biomarker that is highly abundant in normal tissue yet still has a high fold-change, thus increasing its odds of spilling into the circulation. For this reason the table is listed two ways (Table 13a and 13b), one with the left-most column proteins in order of lowest to highest abundance in benign tissue, the other with proteins listed in order of highest to lowest.

In phase one of this study protein DJ-1 displayed a moderate, but significant, fold-change (1.38), and was increased in 13 out of 17 patients. In phase two of this study it was discovered that DJ-1 was higher in the plasma of breast cancer patients (n=48) than that of healthy controls (n=92) (146.5 vs. 74.3, p=0.002). Sensitivity and specificity for elevated DJ-1 as a marker of invasive breast cancer at levels optimized by the ROC analysis were 75% and 84% in an unadjusted model, and 69% and 85% in a model adjusted for age, menopausal status, and prior use of hormone replacement therapy. This is surprising and unexpected because no other marker has been shown to be useful in early stage breast cancer.

It was also discovered that DJ-1 was present at higher levels in the plasma of women with ER-negative breast tumors than those with ER-positive tumors. This is a likely explanation for the low fold-change in the original DIGE study. The original DIGE phase included tissue only from women with ER-positive tumors.

The third phase of this study compared pre-operative and post-operative DJ-1 levels before and after surgery to remove a breast tumor. While the differences did not appear to be statistically significant between pre-operative and post-operative DJ-1 levels, nearly all samples either increased or decreased by more than the 8.2 ng/mL standard set by the referent control subject.

In general, if a subject had a single surgery to remove her tumor, and did not have radiation treatment prior to her second blood sample, her DJ-1 levels decreased from pre-operative levels. However, a re-excision or radiation previous to a subject's blood sample being drawn predicted a post-operative increase in plasma DJ-1 (p=0.0023).

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

TABLE 1
Gel-based information for proteins over-abundant in breast tumor tissue compared to matched benign tissue from the same breast,
ordered from highest fold-change to lowest (N = 18 patients, 18 gels)

| | | Increased | Average | | | | | ΔMW | | | |
|---|---|---|---|---|---|---|---|---|---|---|---|
| | Swiss- | in #/18 | Fold- | | | MW | | [predicted | ΔpI from | % | Mascot |
| Protein Name | Prot no. | gels | Change | p-value | Spot no.*† | (kDa) | pI | (kDa)]‡ | predicted‡ | Coverage | Score |
| Transitional ER | P55072 | 13/16 | 2.24 | 0.00072 | 17 | 89.2 | 5.14 | Reference | Reference | 35 | 175 |
| ATPase | | 10/14 | 1.66 | 0.017 | 18 | | | −36.8 | −0.02 | 25 | 87 |
| (Valosin-containing | | | | | | | | | | | |
| protein) | | | | | | | | | | | |
| Lumican | P51884 | 14/18 | 2.21 | 0.0071 | 19 | 36.7 | 6.17 | Reference | Reference | 33 | 81 |
| Peptidyl-prolyl cis- | P62937 | 15/16 | 2.18 | 4.3e−.06 | 20 | 17.9 | 7.82 | Reference | Reference | 38 | 75 |
| trans isomerase B | | 13/14 | 2.18 | 0.00011 | 21 | | | Reference | Reference | 46 | 92 |
| (Rotamase) | | 14/16 | 2.12 | 0.00022 | 22 | | | 0.78 | −0.48 | 53 | 76 |
| (Cyclophilin B) | | | | | | | | | | | |
| Vimentin | P08670 | 13/16 | 2.16 | 9.3e−.005 | 23 | 53.5 | 5.06 | −4.4 | 0.05 | 42 | 369 |
| | | 13/15 | 1.96 | 0.0018 | 24 | | | −1.8 | 0.22 | 34 | 83 |
| | | 15/18 | 1.94 | 6.60e−.05 | 25 | | | −8.8 | 0.10 | 87 | 368 |
| | | 17/18 | 1.90 | 1.4e−.05 | 26 | | | −7.6 | 0.03 | 46 | 125 |
| | | 14/17 | 1.47 | 0.004 | 27 | | | −10.8 | −0.05 | 62 | 174 |
| | | | 1.7 | 0.0011 | Verification | | | | | | |
| Fructose- | P04075 | 16/18 | 2.15 | 8.4E−.05 | 28 | 39.3 | 8.39 | −6.6 | 0.26 | 74 | 269 |
| bisphosphate aldolase A | | 16/17 | 2.01 | 0.00054 | 29 | | | −7.2 | 0.34 | 78 | 269 |
| | | 16/18 | 1.72 | 0.00046 | 30 | | | −8.1 | 0.49 | 71 | 223 |
| | | 15/18 | 1.54 | 0.0089 | 31 | | | −7.4 | 0.39 | 82 | 276 |
| | | | 2.25 | 3.30E−05 | Verification | | | | | | |
| | | | 2.31 | 0.00016 | Verification | | | | | | |
| | | | 2.01 | 0.00047 | Verification | | | | | | |
| Annexin A2 | P07355 | 14/18 | 2.05 | 0.0087 | 32 | 38.5 | 7.56 | — | Reference | 43 | 91 |
| | | 15/18 | 1.99 | 0.00017 | 33 | | | 2.0 | 0.59 | 33 | 68 |
| | | 16/17 | 1.73 | 6.20e−.05 | 34 | | | −3.4 | 0.30 | 36 | 73 |
| | | | | | | | | −0.80 | | | |
| Leukocyte elastase | P30740 | 15/17 | 1.96 | 0.00019 | 35 | 42.7 | 5.90 | −4.1 | −0.50 | 44 | 107 |
| inhibitor (serpin B1) | | | | | | | | | | | |
| Rho GDP- | P52565 | 18/18 | 1.95 | 2.3e−.07 | 36 | 23.1 | 5.03 | −1.7 | 0.41 | 72 | 151 |
| dissociation inhibitor 1 | | 16/18 | 1.45 | 0.00071 | 37 | | | | | 45 | 76 |
| | | | 1.90 | 1.50e−06 | Verification | | | | | | |
| Fibrinogen beta chain | P02675 | 14/18 | 1.87 | 0.00023 | 38 | 50.8 | 7.95 | −19.0 | −1.04 | 25 | 68 |
| Aconitate hydratase, | Q99798 | 13/17 | 1.86 | 0.000584 | 39 | 82.4 | 6.85 | Reference | Reference | 15 | 66 |
| mitochondrial | | | | | | | | | | | |
| Coactosin-like | Q14019 | 15/18 | 1.85 | 7.9e−.06 | 40 | 15.8 | 5.55 | 2.2 | 0.24 | 73 | 93 |
| protein | | | 2.13 | 6.50e−07 | Verification | | | | | | |
| Chloride intracellular | O00299 | 15/18 | 1.84 | 5.0e−.06 | 41 | 26.8 | 5.09 | −1.8 | −0.02 | 75 | 136 |
| channel protein 1 | | | 1.82 | 0.00023 | Verification | | | | | | |
| Cathepsin D | P07339 | 16/18 | 1.83 | 0.00026 | 42 | 37.9 | 5.60 | Reference | Reference | 41 | 84 |
| | | | 1.78 | 0.0015 | Verification | | | | | | |
| | | | 1.57 | 0.00061 | Verification | | | | | | |
| Annexin A1 | P04083 | 15/18 | 1.82 | 5.4e^{-.05} | 43 | 38.6 | 6.64 | 16 | 1.84 | 50 | 75 |
| 14-3-3 protein | P62258 | 15/16 | 1.75 | 8.60e^{-.05} | 44 | 29.2 | 4.63 | Reference | Reference | 57 | 138 |
| epsilon | | | | | | | | | | | |
| SH3 domain-binding | O75368 | 17/18 | 1.72 | 7.20e^{0.05} | 45 | 12.8 | 5.22 | −13.7 | 0.28 | 90 | 152 |
| glutamic acid-rich-like protein | | 12/18 | 1.69 | 0.0027 | 46 | | | Reference | Reference | 83 | 86 |
| Elongation Factor Tu, | P49411 | 5/5 | 1.72 | 0.0039 | 47 | 45.0 | 6.31 | −7.5 | −0.73 | 55 | 217 |
| mitochondrial | | | | | | | | | | | |
| precursor | | | | | | | | | | | |
| Phosphoglycerate | P18669 | 15/18 | 1.71 | 0.0023 | 48 | 28.7 | 6.75 | −0.53 | 0.57 | 55 | 131 |
| mutase 1 | | | 1.72 | 0.0020 | Verification | | | | | | |
| S-Formylglutathione | P10768 | 14/16 | 1.70 | 0.0014 | 49 | 31.5 | 6.54 | −4.5 | −0.62 | 32 | 92 |
| hydrolase (Esterase D) | | | | | | | | | | | |
| Tubulin beta-5 | P07437 | 14.18 | 1.68 | 0.0034 | 50 | 49.7 | 4.78 | −17.6 | −0.05 | 75 | 175 |
| Myosin light | P60660 | 14/18 | 1.67 | 0.0012 | 51 | 16.8 | 4.56 | 3.0 | 0.02 | 58 | 70 |
| polypeptide 6 | | 13/17 | 1.59 | 0.0015 | 52 | | | Reference | Reference | 72 | 97 |
| | | | 1.60 | 0.0280 | Verification | | | | | | |
| Isocitrate | O75874 | 15/18 | 1.67 | 0.01 | 53 | 46.7 | 6.53 | Reference | Reference | 43 | 136 |
| dehydrogenase, | | | | | | | | | | | |
| cytoplasmic (soluble) | | | | | | | | | | | |
| Phosphatidylethanolamine- | P30086 | 16/18 | 1.65 | 0.00044 | 54 | 20.9 | 7.43 | −.03 | −0.32 | 82 | 123 |
| binding protein | | | | | | | | | | | |
| 1 (Prostatic-binding | | | | | | | | | | | |
| protein) | | | | | | | | | | | |
| 14 kDa | Q9NRX4 | 12/16 | 1.61 | 0.0039 | 55 | 13.8 | 5.65 | 1.3 | 0.14 | 52 | 71 |
| phosphohistidine | | | | | | | | | | | |
| phosphatase | | | | | | | | | | | |
| Septin 11 | Q9NVA2 | 11/14 | 1.56 | 0.0024 | 56 | 49.3 | 6.38 | Reference | Reference | 32 | 92 |
| Actin, aortic smooth | P62736 | 13/16 | 1.44 | 0.023 | 57 | 41.8 | 5.24 | −7.1 | 0.05 | 34 | 73 |
| muscle (Alpha-actin- | | | | | | | | | | | |
| 2) | | | | | | | | | | | |
| Cellular retinoic acid- | P29373 | 15/17 | 1.41 | 0.0069 | 58 | 15.6 | 5.43 | Reference | Reference | 76 | 92 |
| binding protein 2 | | | 2.62 | 0.0011 | Verification | | | | | | |
| Flavin reductase | P30043 | 14/18 | 1.41 | 0.048 | 59 | 22.0 | 7.31 | −0.71 | −0.35 | 70 | 113 |
| Isocitrate | P48735 | 13/18 | 1.40 | 0.011 | 60 | 46.6 | 8.32 | −6.2 | 0.35 | 42 | 116 |
| dehydrogenase 2 | | | | | | | | | | | |
| (NADP), | | | | | | | | | | | |
| mitochondrial | | | | | | | | | | | |
| Protein DJ-1 | Q99497 | 13/17 | 1.38 | 0.014 | 61 | 19.9 | 6.33 | −1.7 | 0.15 | 73 | 119 |
| Carbonyl reductase | P16152 | 15/18 | 1.34 | 0.023 | 62 | 30.2 | 8.55 | Reference | Reference | 46 | 99 |
| Proteasome activator | Q06323 | 15/18 | 1.29 | 0.027 | 63 | 28.7 | 5.78 | 1.6 | −0.06 | 60 | 110 |
| complex subunit 1 | | | | | | | | | | | |

TABLE 2
Gel-based information for proteins less abundant in breast tumor tissue compared to matched benign tissue from the same breast,
ordered from highest fold-change to lowest (N = 18 patients, 18 gels)

| | | Decreased | Average | | | | | ΔMW | | | |
|---|---|---|---|---|---|---|---|---|---|---|---|
| | Swiss- | in #/18 | Fold- | | | | | [predicted | ΔpI from | % | Mascot |
| Protein | Prot no. | gels | Change | p-value | Spot no.*† | MW | PI | (kDa)]‡ | predicted‡ | Coverage | Score |
| Calreticulin | P27797 | 15/16 | −2.29 | 0.0011 | 64 | 46.5 | 4.29 | −19.7 | −0.74 | 40 | 126 |
| Ferritin heavy chain | P02794 | 17/18 | −2.24 | 0.017 | 65 | 21.1 | 5.30 | 1.3 | −0.21 | 53 | 72 |
| (Ferritin H subunit) | | | | | | | | | | | |
| (Proliferation-inducing gene 15 | | | | | | | | | | | |
| protein) | | | | | | | | | | | |
| Alpha-1-antitrypsin | P01009 | 17/18 | −1.91 | 0.0079 | 66 | 44.3 | 5.37 | −28.9 | 0.42 | 36 | 127 |
| (Alpha-1 protease | | 14/18 | −2.01 | 0.018 | 67 | | | 7.2 | −0.8 | | |
| inhibitor) (Alpha-1- | | | −2.03 | 0.0024 | Verification | | | | | | |
| antiproteinase) | | | −1.79 | 0.0062 | Verification | | | | | | |
| Programmed cell death protein 6 | O75340 | 15/18 | −1.90 | 0.014 | 68 | 21.9 | 5.16 | 4.0 | 0.28 | 65 | 82 |
| (Probable calcium-binding protein | | | | | | | | | | | |
| ALG-2) | | | | | | | | | | | |
| Immunoglobulin J chain | P01591 | 13/15 | −1.84 | 0.0024 | 69 | 15.6 | 4.62 | −7.1 | −0.42 | 64 | 74 |
| Alpha-2-HS-glycoprotein | P02765 | 17/18 | −1.83 | 0.0014 | 70 | 33.0 | 4.53 | −32.3 | −0.53 | 43 | 81 |
| Alpha-1- | P01011 | 17/18 | −1.82 | 0.00081 | 71 | 45.3 | 5.32 | −32.4 | −0.25 | 32 | 92 |
| Antichymotrypsin | | | | | | | | | | | |
| Serotransferrin | P02787 | 16/17 | −1.68 | 0.0089 | 72 | 75.2 | 6.70 | Reference | Reference | 26 | 84 |
| (Transferrin) | | | −1.90 | 0.042 | Verification | | | | | | |
| (Siderophilin) | | | | | | | | | | | |
| Ig gamma-1 chain C | P01857 | 17/18 | −1.49 | 0.0044 | 73 | 36.6 | 8.46 | −30.3 | 0.68 | 49 | 88 |
| region | | | −1.42 | 0.021 | 74 | | | | | | 76 |

*Spot numbers correspond to numbers on the gel in FIG. 1.
†Verification spots do not appear on the representative gel from the original study
‡A MW and/or PI of ‘Reference’ protein spots were entered into the software to allow the calculation of the MW's and PI's of remaining spots; by definition change in MW and PI would be zero for these spots

TABLE 3
Biological Information for proteins over abundant in cancerous breast tissue compared to adjacent benign tissue.

| Protein | Secreted/Found In Blood | Function |
|---|---|---|
| Aconitate hydratase, mitochondrial | No Evidence | Catalyzes interconversion of citrate to isocitrate in TCA cycle |
| Actin, aortic smooth muscle (Alpha-actin-2) | No Evidence | Major component of the contractile apparatus. Involved in |
| | | cell structure & motility. |
| Actin, cytoplasmic-1 | No Evidence | A non-muscle cytoskeletal actin |
| (Beta-Actin) | | |
| Adenine phosphoribosyltransferase | No Evidence | Catalyzes a salvage reaction that forms amp & is energetically |
| | | less costly than de novo synthesis (deficiency causes 2,8- |
| | | dihydroxyadenine urolithiasis) |
| Annexin A1 (Annexin I) (Lipocortin I) | No Evidence | Regulates phospholipase A2 activity. A |
| (Calpactin II) (Chromobindin-9) (p35) | | calcium/phospholipids-binding protein that promotes |
| (Phospholipase A2 inhibitor) | | membrane fusion & is involved in exocytosis. |
| | | May have anti-inflammatory activity. |
| Annexin A2 (Annexin II) (Lipocortin II) | Secreted | Thought to cross-link plasma membrane phospholipids with |
| (Calpactin I heavy chain) (Chromobindin-8) | | actin and the cytoskeleton and be involved in exocytosis. |
| Carbonyl reductase (NADPH-dependent | No Evidence | Catalyzes the reduction of several carbonyl compounds |
| carbonyl reductase 1) | | including the antitumor anthracycline antibiotics. Converts |
| | | prostaglandin e2 to prostaglandin f2-alpha |
| Cathepsin D [Contains: Cathepsin D light | No Evidence | Acid protease involved in intracellular protein breakdown, cell |
| chain; Cathepsin D heavy chain] | | invasion, apoptosis |
| Cellular retinoic acid-binding protein 2 | No Evidence | Induces cell differentiation and may antagonize cancer |
| (Cellular retinoic acid-binding protein II) | | progression. |
| | | May regulate the access of retinoic acid to the nuclear retinoic |
| | | acid receptors to help control differentiation. |
| Coactosin-like protein | None found | A calcium-dependent F-actin binding protein that helps |
| | | regulate the actin cytoskeleton. |
| Chloride intracellular channel protein 1 | None found | Localizes mostly to cell nucleus but has both nuclear & |
| (Nuclear chloride ion channel 27) | | plasma membrane chloride ion channel activity-stabilizes cell |
| | | membrane potential, maintains intracellular pH & cell volume, |
| | | & participates in transport. |
| Elongation Factor Tu, mitochondrial (EF 1 | The gene has been detected in serum | Aids in gtp-dependent binding of aminoacyl-tRNA to |
| alpha1); (Prostate tumor-inducing protein-1) | | ribosomes (a-site) during protein synthesis |
| Fibrinogen beta chain [Contains: | Yes, blood-borne glycoprotein | Aids in platelet aggregation (a cofactor) |
| Fibrinopeptide B] | | |
| Flavin reductase (NADPH-dependent | No Evidence | Electron transfer from pyridine nucleotides to flavins, protects |
| diaphorase) | | cells from oxidative damage |
| Fructose-bisphosphate aldolase A (Muscle-type | The gene has been detected in | Glycolysis & carbohydrate degradation; glycolytic enzyme |
| aldolase) (Lung cancer antigen) | the serum of lung cancer patients. | |
| Glyceraldehyde-3-phosphate dehydrogenase | RNA/DNA corresponding to | Catalyzes a step in carbohydrate metabolism - oxidative |
| | this protein has been found in | phosphorylation of glyceraldehyde-3-phosphate |
| | serum | |
| Heat Shock 70 kDa protein 5 | Autoantibodies against this | Aids in the assembly of multimeric protein complexes inside |
| (glucose-regulated protein, 78 kDa) | protein, have been found in the | the ER |
| | serum of prostate cancer patients | |
| Isocitrate dehydrogenase, cytoplasmic (soluble) | Currently being investigated as | Plays a role in cytoplasmic NADPH production |
| | a serum marker for TB | |
| Isocitrate dehydrogenase 2 (NADP), | No evidence of secretion | Intermediary metabolism & energy production. May associate |
| mitochondrial | | with pyruvate dehydrogenase complex |
| Keratin 7 (sarcolectin) | Found in sera of HIV-postive | Proteins arranged in pairs during differentiation of simple and |
| | patients. | stratified epithelial tissues |
| Keratin, type I cytoskeletal 19 (Cytokeratin-19) | A fragment of cytokeratin 19 | — |
| | (CYFRA21-1) has been shown | |
| | to decrease during | |
| | chemotherapy in pt's with | |
| | NSCLC. | |
| Keratin, type II cytoskeletal 8 (Cytokeratin-8) | Autoantibodies to CK-8 have | — |
| (Keratin-8) | been detected in serum | |
| Leukocyte elastase inhibitor (serpin B1); | Has been found in BALF - is | Regulates activity of the neutrophil proteases: elastase, |
| (Monocyte/neutrophil elastase inhibitor) | also on the surface of | cathepsin, and proteinase-3 |
| | neutrophils | |
| Lumican | Yes | May organize collagen fibrils and circumferential growth, |
| | | corneal transparency, and epithelial cell migration & tissue |
| | | repair |
| Malate dehydrogenase, mitochondrial | None found | Plays a role in the malate-aspartate shuttle that operates in the |
| | | metabolic coordination between cytosol & mitochondria |
| Myosin light polypeptide 6 (Myosin light chain | None found | This is the non-muscle and smooth muscle variant of myosin |
| alkali 3) (Myosin light chain 3) | | light chain 6. |
| Peptidyl-prolyl cis-trans isomerase B (PPIase) | Yes | Accelerates the folding of proteins |
| (Rotamase) (Cyclophilin B) | | |
| Phosphatidylethanolamine-binding protein 1 | None found | — |
| (PEBP-1) (Prostatic-binding protein) | | |
| Phosphoglycerate mutase 1 | Found in blood in an anaerobic | Catalyzes reaction of 3-phosphoglycerate to 2- |
| | exercise study | phosphoglycerate in the glycolytic pathway. |
| 14 kDa phosphohistidine phosphatase | None found | Regulates somatic sex differentiation |
| (phosphohistidine phosphatase 1) | | |
| 14-3-3 protein epsilon (14-3-3E | None found | Mediates signal transduction by binding to phosphoserine |
| Tyrosine 3-monooxygenase | | containing proteins |
| Proteasome activator complex subunit 1 | None found | Cleaves peptides (one of three subunits - alpha, beta, gamma). |
| (Proteasome activator 28-alpha subunit) | | Required for efficient antigen processing & |
| | | immunoproteasome assembly |
| Protein DJ-1 (Oncogene DJ1) (Parkinson | Found in serum of Parkinson's | PDJ1 is a positive regulator of androgen receptor-dependent |
| disease protein 7) | Disease patients & controls | transcription. |
| Rho GDP-dissociation inhibitor 1 | None found | Involved in the regulation of the gdp/gtp exchange reaction of |
| (Rho-GDI alpha) | | rho proteins - inhibits dissociation of gdp & binding of gtp |
| S-Formylglutathione hydrolase | None found | The gene has been studied extensively in retinoblastoma |
| Also called Esterase D | | |
| Septin 11 | None found | Potential role in cytokinesis |
| SH3 domain-binding glutamic acid-rich-like | None found | Plays a role in protein-protein interactions in signal |
| protein | | transduction pathways |
| Transitional ER ATPase or | None found | Fragments golgi stacks during mitosis and reassembles them |
| Valosin-containing protein | | after mitosis |
| Tropomyosin alpha-4 chain (Tropomyosin-4) | None found | Binds actin filaments in muscle & non-muscle cells. |
| (TM30p1) | | Associates w/troponin complex. In non-muscle cells |
| | | stabilizes cytoskeleton actin filaments |
| Tubulin beta-5 chain (Beta-tubulin isotype I) | None found | Main component of microtubules. |
| | | Note: Taxanes inhibit microtubule function by stabilizing |
| | | GDP-bound tubulin |
| Vimentin | The endothelial cell-specific | Cytoskeletal element (along w/actins & tubulins) that shows |
| | antibody PAL-E identifies a | mesenchymal specific expression. |
| | secreted form of vimentin in the blood | |
| | vasculature | |

TABLE 4
Biological Information for proteins less abundant in cancerous breast tissue compared to adjacent benign tissue.

| PROTEIN | SECRETED | FUNCTION |
|---|---|---|
| Alpha-1-Antichymotrypsin | Identified as a secreted | Plasma protease inhibitor - deficiency indicative of liver/lung |
| | biomakers of breast cancer. | disease |
| Alpha-1-antitrypsin (Alpha-1 protease | Identified as a secreted | Inhibitor of serine proteases, primary target is elastase. |
| inhibitor) (Alpha-1-antiproteinase) | biomakers of breast cancer. | |
| Alpha-2-HS-glycoprotein | Yes | Endocytosis, brain development, formation of bone tissue. |
| (fetuin A) | | Present in bone marrow hemopoietic matrix. |
| Calreticulin | Yes | Molecular calcium binding chaperone-promotes folding & |
| | | oligomeric assembly in the ER via the calreticulin/calnexin |
| | | cycle |
| Ferritin heavy chain (Ferritin H subunit) | Yes | Stores iron in readily available, non-toxic form |
| (Proliferation-inducing gene 15 protein) | | |
| Ig gamma-1 chain C region | Yes | — |
| Immunoglobulin J chain | Yes | Links two monomer units of either IgM or IgA, also links |
| | | these units to secretory component |
| Programmed cell death protein 6 (Probable | Not listed | Calcium binding protein required for T-cell receptor, -Fas, and |
| calcium-binding protein ALG-2) | | glucocoritcoid-induced cell death |
| Serotransferrin (Transferrin) (Siderophilin) | Yes | Transports iron from sites of absorption and heme degradation |
| (Beta-1-metal-binding globulin) | | to those of storage/utilization. May have a role in stimulating |
| | | cell proliferation. |

TABLE 5
Western Blot results for three proteins

| | | Number | |
|---|---|---|---|
| | | of patients whose | p-value for |
| | Number of | tumor specimens | difference between |
| | patients | were higher than | tumor and adjacent |
| Protein | with blots | normal specimens | normal tissue |
| Peptidyl-prolyl cis- | 17 | 11 | 0.0023 |
| trans isomerase B | | | |
| Rho GDI-alpha | 17 | 10 | 0.005 |

TABLE 6
Characteristics of breast cancer subjects and controls

| | Breast Cancer (n = 48) | Controls (n = 92) | p-value |
|---|---|---|---|
| Mean Age (sd) | 57.5 (12.8) | 51.4 (9.4) | 0.01* |
| ≦40 years | 8% | 10% | |
| 40-50 | 21% | 34% | |
| 50-60 | 23% | 36% | |
| 60-70 | 25% | 16% | |
| 70+ | 23% | 4% | |
| Menopausal Status | | | |
| Pre-menopausal | 33% | 53% | 0.02† |
| Post-menopausal | 67% | 47% | |
| Ever taken HRT | 44% | 27% | 0.04† |

*t-test
†chi-square

TABLE 7
Plasma DJ-1 concentrations and tumor characteristics among 92
controls and among 48 women with newly-diagnosed invasive breast
cancer, according to selected tumor characteristics.

| | | DJ-1 | | |
|---|---|---|---|---|
| | Number | concentrations in ng/ml | | |
| | of subjects | Median | Mean (sd) | p-value |
| Controls | 92 | 58.4 | 74.3 (80.5) | <0.01* |
| All cases | 48 | 121.9 | 146.54 (114.7) | |
| Number of Positive | | | | |
| Lymph Nodes | | | | |
| 0 | 27 | 120.4 | 130.1 | 0.53* |
| 1-3 | 15 | 124.7 | 175.6 | |
| ≧4 | 4 | 121.2 | 128.2 | |
| Tumor Size | | | | |
| ≧2 cm | 25 | 120.4 | 124.19 (61.5) | 0.236* |
| 2-5 cm | 22 | 123.4 | 163.60 (150.5) | |
| Grade | | | | |
| 1 | 11 | 122.3 | 132.7 (58.2) | <0.01*† |
| 2 | 17 | 120.1 | 155.7 (168.1) | |
| 3 | 15 | 125.3 | 141.8 (80.6) | |
| Estrogen Receptor | | | | |
| ER+ | 35 | 121.7 | 136.5 (69.2) | <0.01* |
| ER− | 11 | 124.7 | 185.0 (208.1) | |
| Progesterone Receptor | | | | |
| PR+ | 29 | 121.7 | 134.7 (70.4) | <0.01* |
| PR− | 17 | 122.1 | 171.0 (170.0) | |
| Her2/neu | | | | |
| positive | 5 | 122.1 | 245.8 (306.0) | 0.48* |
| negative | 37 | 121.7 | 134.1 (68.7) | |

*ANOVA
†Kruskal-Wallis test for comparing medians

TABLE 8
Logistic Regression analysis of DJ-1 concentration as a predictor
of case status, for 48 breast cancer cases vs. 92 controls, with and
without adjustment for age.

| | Tertile | Tertile | Tertile 3 |
|---|---|---|---|
| | (<56.50 | (56.50-96.49 ng/ml) | (≧96.50 ng/ml) |
| | ng/ml) | (OR, 95% CI) | (OR, 95% CI) |
| Univariate Model | 1.00 | 9.2 (1.9 to 43.5) | 55.3 (11.6 to 262.5) |
| Adjusted for Age | 1.00 | 7.5 (1.5 to 36.3) | 55.3 (11.3 to 268.9) |
| Adjusted for Age & | 1.00 | 7.9 (1.6 to 38.2) | 54.4 (11.0 to 268.6) |
| Menopausal Status | | | |
| Adjusted for Age, | 1.00 | 8.7 (1.7 to 42.4) | 57.6 (11.3 to 291.5) |
| Menopausal Status, | | | |
| & HRT | | | |

TABLE 9
Sensitivity and Specificity at DJ-1 cut-off of 89.0 ng/ml

| | | | | Model 4 |
|---|---|---|---|---|
| | | | Model 3 | Adjusted |
| | Model 1 | Model 2 | Adjusted for Age | for Age, |
| | Unadjusted | Adjusted | & Menopausal | Menopausal |
| | Model | for Age | Status | Status, & HRT |
| Sensitivity | 79.1% | 77.0% | 72.9% | 68.7% |
| Specificity | 83.7% | 80.4% | 81.5% | 84.7% |
| Accuracy | 85.0% | 82.2% | 81.5% | 81.0% |

TABLE 10
Pre- and Post-Operative DJ-1 values (N = 17)

| | Pre-op | Post-op | p-value paired t-test |
|---|---|---|---|
| Mean | 120.0 (60.4) | 138.1 ng/ml (132.0) | 0.5436 |
| (sd) | | | |
| Median | 109.0 | 89.9 ng/ml | |
| Range | 38.8 to 290.0 | 31.7 ng/ml to 499.3 ng/ml | |

TABLE 11
Table of Change in DJ-1 from Pre-op to Post-op
(Pre-operative DJ-1 minus Post-operative DJ-1) (N = 17)

| | |
|---|---|
| Mean (sd) | −18.3 ng/ml (119.79) |
| Median | −2.4 ng/ml |
| Range | −391.6 ng/ml to 126.0 ng/ml |

TABLE 12
Tumor and Patient Information According to Whether or Not a
Subject's Post-Operative DJ-1 Value Decreased

| | | Patients | |
|---|---|---|---|
| | Patients whose | whose post-op | p-value |
| | post-op value | value did not | Fisher's |
| | decreased (8%) | decrease (9%) | Exact |
| Grade | | | |
| 1 | 3 (42.9) | 1 (11.1%) | |
| 2 | 1 (14.3) | 4 (44.4%) | |
| 3 | 3 (42.9) | 4 (44.4%) | 0.061 |
| Tumor Size | | | |
| <=2 cm | 4 (50%) | 7 (77.8%) | |
| >2 cm | 4 (50%) | 2 (22.2%) | 0.335 |
| Estrogen Receptor | | | |
| ER+ | 5 (62.5%) | 6 (66.7%) | 1.00 |
| Progesterone Receptor | | | |
| PR+ | 2 (25%) | 5 (55.6%) | 0.335 |
| Her2/neu | | | |
| positive | 6 (75%) | 6 (75%) | 1.00 |
| Menopause | | | |
| Pre | 4 (50%) | 1 (11.1%) | |
| Post | 4 (50%) | 8 (88.9%) | 0.131 |
| Second Surgery or | | | |
| Radiation before post-op | | | |
| Blood draw | | | |
| Yes | 0 (0%) | 7 (77.8%) 2 (22.2%) | 0.0023 |
| No | 8 (100%) | | |

TABLE 13a
Proteins close to the top theoretically have the most desirable characteristics for a future biomarker with high specificity

| Lowest to Highest abundance in BENIGN | Ordered from Highest to lowest FOLD- | Ordered from MOST CONSISTENT TO LEAST |
|---|---|---|
| tissue | CHANGE | CONSISTENT |
| Lumican | Tropomyosin alpha-4 chain* | Tropomyosin alpha-4 chain* |
| (secreted protein) | | |
| Protein DJ-1 (Secreted Protein) ELISA KIT | Lumican (secreted protein) | Annexin A2 (secreted protein) AB |
| Coactosin-like protein* | Peptidyl-prolyl cis-trans isomerase B | Peptidyl-prolyl cis-trans isomerase B |
| 14-3-3 protein epsilon | Annexin A2 (secreted protein) AB | 14-3-3 protein epsilon |
| Rho GDP-dissociation inhibitor 1* | Rho GDP-dissociation inhibitor 1* | SH3 domain binding glutamic acid-rich-like protein |
| Chloride intracellular channel protein 1* | Coactosin-like protein* | Rho GDP-dissociation inhibitor 1* |
| Myosin light polypeptide 6 * | Chloride intracellular channel protein 1* | Coactosin-like protein* |
| Peptidyl-prolyl cis-trans isomerase B | 14-3-3 protein epsilon | Chloride intracellular channel protein 1* |
| precursor | | |
| Tropomyosin alpha-4 chain* | SH3 domain binding glutamic acid-rich- | Lumican (secreted protein) |
| | like protein | |
| Annexin A2 (secreted protein) AB | Myosin light polypeptide 6 * | Myosin light polypeptide 6 * |
| SH3 domain binding glutamic acid-rich-like | Protein DJ-1 (Secreted Protein) | Protein DJ-1 (Secreted Protein) |
| protein | ELISA KIT | ELISA KIT |

TABLE 13b
Proteins close to the top theoretically have the most desirable characteristics for a future biomarker with high sensitivity

| Highest to lowest abundance in BENIGN | Ordered from Highest to lowest FOLD- | Ordered from MOST CONSISTENT TO LEAST |
|---|---|---|
| tissue | CHANGE | CONSISTENT |
| SH3 domain binding glutamic acid-rich-like | Tropomyosin alpha-4 chain* | Tropomyosin alpha-4 chain* |
| protein | | |
| Annexin A2 (secreted protein) AB | Lumican (secreted protein) | Annexin A2 (secreted protein) AB |
| Tropomyosin alpha-4 chain* | Peptidyl-prolyl cis-trans isomerase B | Peptidyl-prolyl cis-trans isomerase B |
| Peptidyl-prolyl cis-trans isomerase B | Annexin A2 (secreted protein) AB | 14-3-3 protein epsilon |
| precursor | | |
| Myosin light polypeptide 6 * | Rho GDP-dissociation inhibitor 1* | SH3 domain binding glutamic acid-rich-like protein |
| Chloride intracellular channel protein 1* | Coactosin-like protein* | Rho GDP-dissociation inhibitor 1* |
| Rho GDP-dissociation inhibitor 1* | Chloride intracellular channel protein 1* | Coactosin-like protein* |
| 14-3-3 protein epsilon | 14-3-3 protein epsilon | Chloride intracellular channel protein 1* |
| Coactosin-like protein* | SH3 domain binding glutamic acid-rich- | Lumican (secreted protein) |
| | like protein | |
| Protein DJ-1 (Secreted Protein) ELISA KIT | Myosin light polypeptide 6 * | Myosin light polypeptide 6 * |
| Lumican (secreted protein) | Protein DJ-1 (Secreted Protein) ELISA | Protein DJ-1 (Secreted Protein) ELISA KIT |
| | KIT | |

What is claimed:

1. A method for determining the presence of breast cancer in a subject, said method comprising determining the level of a panel of biomarkers in a fluid sample of the subject, wherein the subject is determined to have breast cancer if the level of biomarkers in the fluid sample of the subject is statistically different from the level of the biomarkers that has been associated with normal controls, and wherein the panel of biomarkers comprises at least three biomarkers selected from the group consisting of SH3 domain binding glutamic acid-rich-like protein; annexin A2; tropomyosin alpha-4 chain; peptidyl-prolyl cis-trans isomerase B; myosin light polypeptide 6; chloride intracellular channel protein 1; Rho GDP-dissociation inhibitor 1; 14-3-3 protein epsilon; coactosin-like protein; lumican; and DJ-1 protein.

2. The method of claim 1, wherein the panel of biomarkers comprises DJ-1 protein.

3. The method of claim 1, wherein the subject is determined to have breast cancer if the level of biomarkers in the patient sample is statistically more similar to the level of the biomarkers that has been associated with breast cancer than the level of the biomarkers that has been associated with the normal controls.

4. The method of claim 1, wherein said method comprises determining the level of at least five biomarkers.

5. The method of claim 1, wherein said method comprises determining the level of at least six biomarkers.

6. The method of claim 1 further comprising comparing the level of biomarkers determined in the fluid sample to a level of biomarkers that has been associated with breast cancer and a level of biomarkers that has been associated with normal controls.

7. A kit for determining the presence of breast cancer, said kit comprising assay kits for determining the level of a panel of biomarkers, wherein said panel of biomarkers comprises at least three biomarkers selected from the group consisting of: SH3 domain binding glutamic acid-rich-like protein; annexin A2; tropomyosin alpha-4 chain; peptidyl-prolyl cis-trans isomerase B; myosin light polypeptide 6; chloride intracellular channel protein 1; Rho GDP-dissociation inhibitor 1; 14-3-3 protein epsilon; coactosin-like protein; lumican; and DJ-1 protein.

8. The kit of claim 7, wherein said kit comprises assay kits for determining the level of at least five biomarkers.

9. The kit of claim 7, wherein said kit comprises assay kits for determining the level of at least six biomarkers.

10. The kit of claim 7, wherein said kit comprises an assay kit for determining the level of DJ-1 protein.

11. A method for determining the presence of breast cancer in a subject, said method comprising determining the level of a panel of biomarkers in a fluid sample of the subject, wherein the subject is determined to have breast cancer if the level of biomarkers in the fluid sample of the subject is statistically different from the level of the biomarkers that has been associated with normal controls, and wherein the panel of biomarkers comprises DJ-1 protein and a biomarker selected from the group consisting of SH3 domain binding glutamic acid-rich-like protein; annexin A2; tropomyosin alpha-4 chain; peptidyl-prolyl cis-trans isomerase B; myosin light polypeptide 6; chloride intracellular channel protein 1; Rho GDP-dissociation inhibitor 1; 14-3-3 protein epsilon; coactosin-like protein; lumican; and a combination thereof.

12. The method of claim 11, wherein the panel of biomarkers comprises DJ-1 protein and at least two biomarkers selected from the group consisting of SH3 domain binding glutamic acid-rich-like protein; annexin A2; tropomyosin alpha-4 chain; peptidyl-prolyl cis-trans isomerase B; myosin light polypeptide 6; chloride intracellular channel protein 1; Rho GDP-dissociation inhibitor 1; 14-3-3 protein epsilon; coactosin-like protein; and lumican.

13. The method of claim 11, wherein the panel of biomarkers comprises DJ-1 protein and at least three biomarkers selected from the group consisting of SH3 domain binding glutamic acid-rich-like protein; annexin A2; tropomyosin alpha-4 chain; peptidyl-prolyl cis-trans isomerase B; myosin light polypeptide 6; chloride intracellular channel protein 1; Rho GDP-dissociation inhibitor 1; 14-3-3 protein epsilon; coactosin-like protein; and lumican.

14. The method of claim 11, wherein the panel of biomarkers comprises DJ-1 protein and at least four biomarkers selected from the group consisting of SH3 domain binding glutamic acid-rich-like protein; annexin A2; tropomyosin alpha-4 chain; peptidyl-prolyl cis-trans isomerase B; myosin light polypeptide 6; chloride intracellular channel protein 1; Rho GDP-dissociation inhibitor 1; 14-3-3 protein epsilon; coactosin-like protein; and lumican.

15. The method of claim 11, wherein the panel of biomarkers comprises DJ-1 protein and at least five biomarkers selected from the group consisting of SH3 domain binding glutamic acid-rich-like protein; annexin A2; tropomyosin alpha-4 chain; peptidyl-prolyl cis-trans isomerase B; myosin light polypeptide 6; chloride intracellular channel protein 1; Rho GDP-dissociation inhibitor 1; 14-3-3 protein epsilon; coactosin-like protein; and lumican.
